(19) United States
(12) Patent Application Publication
(10) Pub. No.: US 2018/0261325 A1
(43) Pub. Date: Sep. 13, 2018

(54) SYSTEMS AND METHODS FOR PROVIDING AGGREGATED AND CUSTOMIZABLE CLINICAL DECISION SUPPORT INFORMATION

(71) Applicant: Merge Healthcare Incorporated, Chicago, IL (US)

(72) Inventor: Pete Nanos, Chicago, IL (US)

(21) Appl. No.: 15/533,992

(22) PCT Filed: Apr. 22, 2016
(86) PCT No.: PCT/US16/28990

§ 371 Date: Jun. 7, 2017

(51) Int. Cl. G16H 50/20; G16H 50/70; G16H 20/40; G16H 10/60; G06N 5/02

(52) U.S. Cl. CPC G16H 50/20; G16H 50/70; G16H 20/17; G16H 10/60; G06N 5/02; G16H 20/40

(57) ABSTRACT

Methods, systems, and machine-readable mediums for providing clinical appropriateness information. One system includes a server and a plurality of clinical decision support (“CDS”) systems. Each CDS system includes a portal accessible over a communication network. The server includes an electronic processor. The electronic processor is configured to receive input, identify a set of CDS systems, and for each CDS system included in the set of CDS systems, establish electronic communication with the portal included in the CDS system, submit a request for appropriateness information to the CDS system based on the input through the portal, and receive appropriateness information from the CDS system. The electronic processor is also configured to aggregate the appropriateness information received from each CDS system included in the set of CDS systems to generate an aggregated list and display the aggregated list on a display device.

### FIELD

Embodiments of the invention relate to systems and methods for receiving clinical appropriateness information from each of a plurality of clinical decision support systems to aid medical procedure selection and ordering.

### BACKGROUND

A physician may use a clinical data collection system, such as an electronic health record (“EHR”), to document clinical data for a patient. In some embodiments, the physician may use the data collection system or a separate system, such as a computerized physician order entry (“CPOE”) system, to order one or more medical procedures for a patient. However, a physician may order an unnecessary or sub-optimal procedure for a patient, which may put the patient's health at risk and wastes medical resources.

### SUMMARY

Accordingly, embodiments of the invention provide systems and methods for integrating a clinical decision support (“CDS”) system with a clinical data collection system, such as an EHR system. For example, one embodiment provides a system that includes a clinical data collection system that stores clinical data for a patient and receives a selection of a medical procedure. The clinical data collection system interfaces (e.g., over one or more computer networks) with a plurality of CDS systems. In particular, each CDS system may provide a portal accessible over a communication network, such as the Internet, to authorized devices and users. The clinical data collection system interfaces with these portals and provides the CDS systems with clinical data for the patient and an identifier of the selected medical procedure. Each CDS system returns appropriateness information, such as an appropriateness indicator (e.g., a score), to the clinical data collection system. The appropriateness information indicates the clinical appropriateness of the selected medical procedure based on the clinical data. Alternatively or in addition, the appropriateness information includes a recommended medical procedure.

The clinical data collection system creates a user interface that displays the appropriateness information received from the CDS systems to a user in a format that allows the user to compare and contrast the appropriateness information. In some embodiments, the user interface displays received appropriateness information with an identifier of the CDS system that provided the appropriateness information to allow a user to weigh the source of the appropriateness information.

In some embodiments, the clinical data collection system manages a list of a plurality of CDS systems that are configured to provide appropriateness information to the clinical data collection system. The list may include an identifier of each CDS system and information for accessing each CDS system (e.g., an address of a portal provided by the CDS system, credentials for accessing the CDS system, a format for submitting data to the CDS system, a format for receiving output from the CDS system, and the like). Accordingly, to add or remove available CDS systems, the list may be updated. Therefore, the CDS systems accessed by the clinical data collection system are customizable (e.g., at a facility level, a physician level, a patient level, and the like). For example, in some embodiments, the clinical data collection system provides a user interface where a user may add, remove, activate, and deactivate CDS systems from the list, which allows the clinical data collection system to dynamically adjust to the requirements of a particular facility, physician, patient, and the like.

For example, one embodiment provides a system for providing clinical appropriateness information. The system includes a plurality of clinical decision support (“CDS”) systems. Each of the plurality of CDS systems includes a portal allowing devices external to the CDS system to access the CDS system over a communication network. The system also includes a server including one or more non-transitory, machine-readable mediums, an electronic processor, and an input/output interface for accessing each of the plurality of CDS systems over the communication network. The machine-readable medium storing a list of the plurality of CDS systems including data for accessing each of the plurality of CDS systems through the portal included in each of the plurality of CDS systems. The electronic processor is configured to receive input, and identify, based on the list of the plurality of CDS systems, a set of CDS systems. For each CDS system included in the set of CDS systems, the electronic processor is also configured to establish electronic communication with the portal included in the CDS system, submit a request for appropriateness information to the CDS system based on the input through the portal, and receive appropriateness information from the CDS system. The electronic processor is also configured to aggregate the appropriateness information received from each CDS system included in the set of CDS systems to generate an aggregated list and display the aggregated list on a display device.

Another embodiment provides a method of providing clinical appropriateness information generated by a plurality of clinical decision support (“CDS”) systems. The method includes receiving input, and identifying, with an electronic processor, a set of CDS systems to access from the plurality of CDS systems. The method also includes, for each CDS system included in the set of CDS systems, establishing, with the electronic processor, electronic communication with the CDS system, submitting, with the electronic processor, a request for appropriateness information to the CDS system based on the input, and receiving, with the electronic processor, appropriateness information from the CDS system. In addition, the method includes aggregating, with the electronic processor, the appropriateness information received from each CDS system included in the set of CDS systems to generate an aggregated list and displaying the aggregated list on a display device.

Yet another embodiment provides one or more non-transitory machine-readable mediums containing instructions that, when executed by one or more electronic processors, perform a set of functions. The set of functions includes automatically receiving input from a clinical data collection system, the input including clinical data for a patient and order data specifying a medical procedure ordered for the patient and identifying a configurable set of clinical decision support (“CDS”) systems to access from a plurality of CDS systems. The set of functions also includes, for each CDS system included in the set of CDS systems, establishing electronic communication with the CDS system through a portal included in the CDS system over a communication network, submitting a request for appropriateness information to the CDS system based on the input, and receiving appropriateness information from the CDS system, the appropriateness information includes at least one selected from the group consisting of an indicator of an appropriateness of the medical procedure ordered for the patient based on the clinical data and a recommended medical procedure for the patient based on the clinical data. The set of functions also including aggregating the appropriateness information received from each CDS system included in the set of CDS systems to generate an aggregated list and displaying the aggregated list on a display device, the aggregated list including a ranking of the appropriateness received from each CDS system included in the set of CDS systems.

Other aspects of the invention will become apparent by consideration of the detailed description and accompanying figures.

### BRIEF DESCRIPTION OF THE DRAWINGS

FIG. 1 illustrates a system for providing clinical appropriateness information.

FIG. 2 illustrates a method performed by the system of FIG. 1 for providing clinical appropriateness information.

### DETAILED DESCRIPTION

Before embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways.

Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and may include electrical connections or couplings, whether direct or indirect. Also, electronic communications and notifications may be performed using any known means including direct connections, wireless connections, etc.

A plurality of hardware and software based devices, as well as a plurality of different structural components may be utilized to implement the invention. In addition, it should be understood that embodiments of the invention may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects of the invention may be implemented in software (e.g., stored on non-transitory machine-readable medium) executable by one or more processors. As such, it should be noted that a plurality of hardware and software based devices, as well as a plurality of different structural components, may be utilized to implement the invention. For example, “mobile device,” “computing device,” and “server” as described in the specification may include one or more electronic processors, one or more memory modules including non-transitory machine-readable medium, one or more input/output interfaces, and various connections (e.g., a system bus) connecting the components.

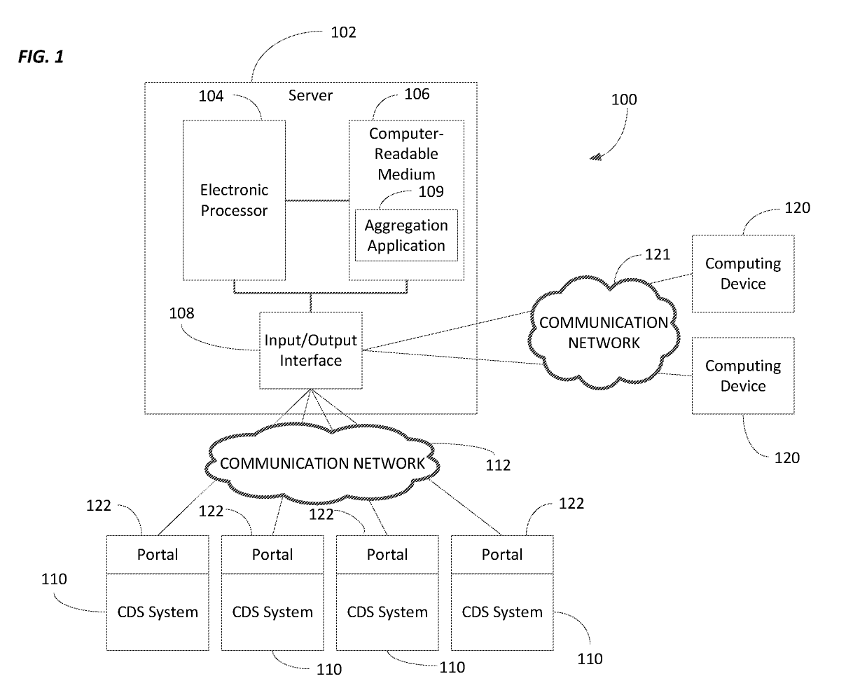

FIG. 1 illustrates a system 100 for providing clinical appropriateness information. The system 100 includes a server 102. The server 102 includes an electronic processor 104 (e.g., microprocessor, application specific integrated circuit, or other suitable electronic device), non-transitory machine-readable medium 106, and an input/output interface 108. It should be understood that the server 102 may also include additional components and may be configured to perform additional functionality than the functionality described herein. For example, in some embodiments, in addition to aggregating clinical appropriateness information as described herein, the server 102 may be configured to document clinical data for a patient (e.g., an electronic health record (“EHR”) system) and/or order medical procedures for a patient (e.g., a computerized physician order entry (“CPOE”) system). Accordingly, the server 102 may be part of a clinical data collection system. In addition, the functionality performed by the server 102 as described below may be distributed among multiple devices. Thus, functionality described herein as being performed by the server 102 or, more particularly, the electronic processor 104, may be performed by one or more electronic processors included in the server 102 or remote from the server 102.

The machine-readable medium 106 (e.g., read-only memory, random-access memory, or combinations thereof) stores instructions and data. In particular, the machine-readable medium 106 stores an aggregation application 109 that includes a plurality of instructions and associated data. The electronic processor 104 retrieves instructions from the machine-readable medium 106, including the aggregation application 109, and executes the instructions to perform a set of functions including the methods described herein.

The input/output interface 108 receives data from and transmits data to devices and networks external to the server 102. For example, as illustrated in FIG. 1, the server 102 communicates with a plurality of clinical decision support (“CDS”) systems (referred to individually as a CDS system 110 or collectively as CDS systems 110) through the input/output interface 108. In some embodiments, the server 102 communicates with one or more of the CDS systems 110 over a wired or wireless communication network 112 (e.g., the Internet, a local area network (“LAN”), and the like). In other embodiments, the server 102 may communicate with one or more of the CDS systems 110 over a dedicated connection. As illustrated in FIG. 1, the server 102 may also communicate (e.g., through the input/output interface 108) with one or more external computing devices 120 (e.g., laptop computers, desktop computers, terminals, tablet computers, smart telephones, smart watches, smart televisions, and the like). In some embodiments, the server 102 communicates with one or more of the external computing devices 120 over a wired or wireless communication network 121 (e.g., the Internet, a LAN, and the like). In other embodiments, the server 102 may communicate with one or more of the external computing devices 120 over a dedicated connection. It should be understood that, in some embodiments, the aggregation application 109, or a portion thereof, is executed by an electronic processor included in a computing device 120. For example, in some embodiments, the aggregation application 109 is executed by an electronic processor included in a computing device 120, and the computing device 120 is configured to communicate with the plurality of CDS systems 110 over a wired or wireless communication network (e.g., the Internet, a local area network (“LAN”), and the like) or a direct connection. In other words, the aggregation application 109 may be executed as a hosted application, a local application, or a combination thereof.

Each of the plurality of CDS systems 110 provides information to assist a healthcare professional with clinical decision-making tasks, such as ordering medical procedures for a patient. In some embodiments, a CDS system 110 is configured to review patient information and an order for a selected medical procedure for the patient and determine whether the selected medical procedure is appropriate for the patient. In particular, a CDS system 110 may provide an indicator, such as a score, color-coding, and the like, designating the appropriateness of the selected medical procedure for the patient and, optionally, may recommend one or more other procedures that may be more appropriate (i.e., be associated with better scores given the information contained in the order (e.g., compliant, history, related test results, patient information) and/or information relating to the patient). Similarly, a CDS system 110 may be configured to review patient information and generate a set of proposed diagnoses. A healthcare professional may review the set of proposed diagnoses to generate a final diagnosis for a patient or order further tests to narrow down the set of diagnoses. In some embodiments, the set of proposed diagnoses also specify what tests would be useful to confirm each diagnosis. Also, in some embodiments, a CDS system 110 may alert a healthcare professional of possible drug interactions based on a list of medications taken by a patient and optionally recommend alternative medications.

A CDS system 110 may use a knowledge base and an associated interference engine to apply rules to input data (e.g., patient data). Alternatively or in addition, a CDS system 110 may use artificial intelligence (i.e., machine learning) to find patterns in historical clinical data. These types of CDS systems 110 may use neural networks, support vector machines, and generic algorithms to automatically develop rules or patterns for identifying particular medical conditions (e.g., generating a diagnosis, determining the appropriateness of a particular test, determining potential drug interactions, and the like).

As illustrated in FIG. 1, each CDS system 110 may include a portal 122 that allows external devices to input data and receive output from the CDS system 110. In some embodiments, the portal 122 includes a web page that a device may access through a browser application. The portal 122 may prompt for and receive credentials (e.g., a username and a password) to ensure that only authorized users are accessing the CDS system 110. For example, in some embodiments, a CDS system 110 may generate one or more graphical user interfaces (“GUIs”) that prompt a user for particular information used by the CDS system 110 (e.g., credentials, patient clinical data, a selected medical procedure, and the like). The output provided by a CDS system 110 may similarly be provided within a GUI. In other embodiments, however, the portal 122 for a CDS system 110 may require that data be submitted in the form of a file or other data object that conforms to a particular format (e.g., a comma delimited text file, a markup language, and the like). Similarly, a CDS system 110 may output data as a file with a predefined format.

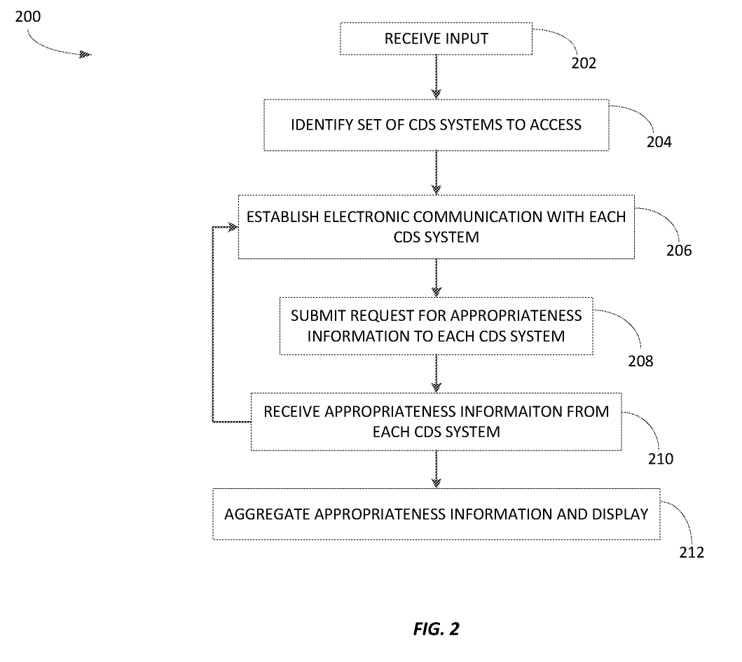

As noted above, the electronic processor 104 executes instructions stored in the machine-readable medium 106 to perform a set of functions. In particular, the electronic processor 104 executes instructions included in the aggregation application 109 stored in the machine-readable medium 106 to provide aggregated clinical appropriateness information to a user. For example, FIG. 2 illustrates a method 200 performed by the electronic processor 104 through execution of the aggregation application 109 to collect and aggregate data from the plurality of CDS systems 110.

As illustrated in FIG. 2, the method 200 includes receiving input representing a request for appropriateness information (at block 202). The input may include clinical data including patient data (e.g., name, demographics, insurance information, and the like) and medical data (e.g., medical history, family history, and the like). The input may also include order data (e.g., a procedure ordered by a healthcare professional, such as the type of procedure, anatomy the procedure is applied to, parameters for the procedure (e.g., contrast or no contrast), and the like). In some embodiments, the aggregation application 109 is configured to generate a GUI to prompt a user for this information. In other embodiments, the aggregation application 109 is executed as part of a clinical data collection system, which may automatically capture all or a portion of this data. Automatically capturing this data eliminates the need for the user to enter information multiple times, which wastes computer resources and introduces potential manual errors. For example, after a user enters order data for a particular patient, the aggregation application 109 may be configured to automatically (or in response to selection of a selection mechanism by the user) capture the data associated with a request for appropriateness information. It should be understood that data associated with a request may be collected from numerous mediums (internal or external to the server 102 or the computing device 120 executing the aggregation application 109).

The method 200 also includes identifying a set of CDS systems included in the plurality of CDS systems to access (at block 204). For example, in some embodiments, the aggregation application 109 is configured to access each of the plurality of CDS systems 110, but a particular healthcare professional may only want to access a subset of the plurality of CDS systems 110. Therefore, the aggregation application 109 may store and maintain a list of the plurality of CDS systems 110 and associated data for accessing each CDS system 110 (e.g., credentials, uniform resource locator, file format, and the like). The list (or a separate data structure) may also include data identifying a set of the plurality of CDS systems 110 associated with a particular user. The set of the plurality of CDS systems 110 may be set at an individual user level (e.g., an individual healthcare professional), a user group level (e.g., a group of individual healthcare professionals), a clinic or hospital level, or a clinic or hospital group level (e.g., a group of clinics and/or hospitals). Accordingly, in these embodiments, the aggregation application 109 may be configured to identify the set of the plurality of CDS systems 110 (e.g., all or a subset of the plurality of CDS systems 110) based on the user providing the input (or the user originally ordering the medical procedure) (e.g., based on a unique user identifier and/or a group, clinic, or hospital the user is associated with). Information identifying a particular user may be included within a user data record stored on the server 102 or an external storage device accessible by the server 102 and may be provided by the user as part of request for appropriateness information. Similarly, the list identifying the plurality of CDS systems 110 and associated information may be stored on the server 102 or a computing device 120 executing the aggregation application 109 or an external device accessible by the server 102 or the computing device 120.

Also, in some embodiments, the set of the plurality of CDS systems 110 is based on the data associated with the request, such as the clinical data or the selected medical procedure. For example, the insurance carried by a particular patient may dictate that one or more particular CDS systems 110 be used. Also, some CDS systems 110 may be tailored for particular types of patients (e.g., particular ages, particular ethnicities, a particular gender, and the like). Similarly, particular CDS systems 110 may be associated with particular medical conditions that may be relevant or irrelevant for a particular patient. For example, a CDS system 110 specializing in diabetic conditions may not be relevant for a patient that is not diabetic. Furthermore, a CDS system 110 may be specialized in particular types of procedures. For example, a CDS system 110 may be specialized for radiological procedures, which may make the CDS system 110 irrelevant for an order including a blood test. As another example, if a user orders a radiological exam for a patient and the patient carries BlueCross insurance, the aggregation application 109 may access one or more CDS systems 110 promoted or required by BlueCross and one or more CDS systems 110 providing customized information for radiological procedures. Again, the rules for identifying the set of CDS systems 110 based on the clinical data (e.g., patient data), order data, or both may be stored on the server or a data storage device external to but accessible by the server 102.

Also, in some embodiments, a user may manually select one or more CDS systems 110 to access. For example, the aggregation application 109 may be configured to display a list of the plurality of CDS system 110 (e.g., representing available systems) to the user submitting the request for appropriateness information, which the user may use to manually select desired CDS systems 110 from the plurality of available CDS systems 110.

Accordingly, using the list and selection mechanisms described above, a network of physicians (e.g., a hospital or a group of hospitals or individuals) may establish a list of available CDS systems 110 (each of which likely require complex negotiations and aggregations with the provider of the CDS system 110) that a network of physicians may modify as desired (e.g., to add new CDS systems 110 or remove existing CDS systems 110) to dictate particular CDS systems 110 for users while still, in some embodiments, allowing a user to manually select desired CDS system 110 for particular orders and optionally customizing CDS systems 110 based on the request (e.g., the user requesting the information or ordering the procedure, clinical data, selected medical procedure, and the like). Also, it should also be understood that, in some embodiments, the aggregation application 109 is configured to generate one or more GUIs that receive user input for configuring selection rules and parameters for selecting the set of CDS systems based on the user making the request, clinical data, order data, and the like. Accordingly, a user may use the aggregation application 109 to both submit a request for appropriateness information and configure the aggregation application 109.

After identifying the set of CDS systems 110 (at block 204), the aggregation application 109 establishes electronic communication with each CDS system 110 included in the identified set (at block 206). As noted above, the aggregation application 109 may access a particular CDS system 110 through a web page provided by the CDS system 110 as a portal 122. Therefore, the aggregation application 109 accesses the web page portal and automatically provides any necessary credentials (e.g., username or password) necessary to access the CDS system 110. In some embodiments, as noted above, the list of available CDS systems 110 may include the credentials for accessing a particular CDS system 110. Therefore, the aggregation application 109 may use the stored credentials to access a CDS system 110. It should be understood that multiple credentials may be stored in the list for a particular CDS system 110. For example, if multiple physicians are allowed to access a particular CDS system 110, the list may include credentials for each physician or credentials shared by the physicians. Therefore, the aggregation application 109 may be configured to select appropriate credentials for the CDS system 110 based on the user making the request. In other embodiments, the aggregation application 109 may prompt the user for credentials, which the aggregation application 109 may use to access a CDS system 110. In some embodiments, the aggregation application 109 may also store credentials received from a user for later use.

After establishing communication with a particular CDS system 110 (at block 206), the aggregation application 109 submits a request for appropriateness information to the CDS system 110 based on the received input (formatting the information as necessary) (at block 208). In particular, as noted above, the aggregation application 109 may submit clinical data associated with a patient and an identifier of a medical procedure selected for the patient to the CDS system 110. It should be understood that, in some embodiments, a CDS system 110 may be configured to automatically retrieve all or a portion of data associated with a request for appropriateness information. For example, in some embodiments, the aggregation application 109 may provide a CDS system 110 with a unique identifier of the patient, which the CDS system 110 may use to retrieve necessary clinical data. Also, in some embodiments, the aggregation application 109 may submit a unique identifier of a medical order, which the CDS system 110 may use to retrieve the associated procedure data and clinical data.

In response to the submitted request (at block 208), the aggregation application 109 receives appropriateness information from the CDS system 110 (at block 210). As noted above, the appropriateness information may include an appropriateness indicator, such as a score. The appropriateness information may also include one or more recommend procedures.

The aggregation application 109 establishes electronic communication with each CDS system 110 included in the identified set of CDS systems 110 (at block 206) and, consequently, receives appropriateness information from each CDS system 110 included in the identified set of CDS systems 110 (at block 210). The aggregation application 109 then aggregates the received appropriateness information and generates an aggregated listing of appropriateness information to aid selection and ordering of a medical procedure, which the aggregation application 109 displays to the user (at block 212). In some embodiments, the aggregated list includes the appropriateness information received from each CDS system 110 with an identifier of the CDS system 110 that provided the appropriateness information. Therefore, in these embodiments, the user may weigh the source of the appropriateness information. Also, in some embodiments, the aggregated list includes aggregated appropriateness information, such as an average score based on the scores received from the CDS systems 110. In addition, in some embodiments, the aggregation application 109 compares the aggregated appropriateness information to one or more thresholds to identify whether the selected medical should be approved. For example, in some embodiments, a user may only select a medical procedure that is associated with an average appropriateness score that exceeds a predefined threshold (e.g., without requiring an additional level of approval).

The aggregated list may be displayed on a display device (e.g., a touchscreen, liquid crystal device (“LCD”) device, and the like) connected to the server 102 (e.g., through the input/output interface 108) or one of the external computing devices 120. For example, as described above, an external computing device 120 may access the functionality provided by the aggregation application 109 executed by the server 102 through a browser application, and, similarly, may receive the aggregated list through the browser application (e.g., as part of a GUI generated by the aggregation application 109).

In some embodiments, the aggregation application 109 also tracks actions taken by a user in response to the displayed aggregated appropriateness information. For example, when a user modifies an order based on the appropriateness information provided by a particular CDS system 110, the aggregation application 109 stores data tracking this change. Similarly, in some embodiments, the aggregation application 109 receives user input selecting the appropriateness information from one CDS system 110 (e.g., a preferred CDS system). Accordingly, the aggregation application 109 may store this input to track what CDS systems 110 are most preferred or used. The aggregation application 109 may also use this stored input to designate rankings for CDS systems 110 (e.g., what CDS systems 110 are most often preferred or used). All or a portion of this information may be presented to the user by the aggregation application 109 to aid the user in selecting a particular medical procedure.

As noted above, the aggregation application 109 may be used as part of a clinical data collection system. Therefore, in some embodiments, the aggregated appropriateness information may be displayed within an EHR system or an ordering system along with insurance approval or payment information (e.g., whether or how much of the procedure will be covered by one or more insurance providers).

In some embodiments, after helping a user (e.g., an ordering provider) make an informed choice for a patient using aggregated information from the plurality of CDS systems 110 as described above, the aggregation application 109 may use received patient data to determine whether a patient is associated (e.g., is a covered life) with a radiology business management (“RBM”) partner or payor partner. When the patient is associated with such a partner, the aggregation application 109 submits the order data that the aggregation application 109 helped select to the appropriate partner system. The partner system may then run the order data through one or more additional CDS systems 110 to take account of their own self-developed acceptable use criteria (“AUC”) in determining when an exam or procedure is appropriate and, thus, something the partner or their customers (i.e., the payor customers) will pay for. When the partner system determines that the ordered procedure is appropriate, the partner system may provide an authorization for the order data (e.g., an authorization number) to the aggregation application 109. The aggregation application 109 may then submit the order data as advised by one or more CDS systems and approved by any applicable partner application, such as by submitting the order data to a RIS for processing.

Thus, embodiments of the invention provide an aggregation application that provides aggregated appropriateness information from multiple CDS systems by automatically accessing one or more CDS system from a customizable list of available CDS systems, enter the required information for accessing the CDS system, obtain the resulting appropriate information, and provide the aggregated information in a format that allows a user to easily compare and contrast the information. Thus, embodiments of the invention eliminate manual errors and reduce processing time for receiving appropriateness information.

Various features and advantages of the invention are set forth in the following claims.

What is claimed is:

1. A system for providing clinical appropriateness information, the system comprising:
a plurality of clinical decision support (“CDS”) systems, each of the plurality of CDS systems including a portal allowing devices external to the CDS system to access the CDS system over a communication network; and
a server including one or more non-transitory, machine-readable mediums, an electronic processor, and an input/output interface for accessing each of the plurality of CDS systems over the communication network, the machine-readable medium storing a list of the plurality of CDS systems including data for accessing each of the plurality of CDS systems through the portal included in each of the plurality of CDS systems, the electronic processor configured to:
receive input,
identify, based on the list of the plurality of CDS systems, a set of CDS systems,
for each CDS system included in the set of CDS systems,
establish electronic communication with the portal included in the CDS system,
submit a request for appropriateness information to the CDS system based on the input through the portal, and
receive appropriateness information from the CDS system, and
aggregate the appropriateness information received from each CDS system included in the set of CDS systems to generate an aggregated list and display the aggregated list on a display device.

2. A method of providing clinical appropriateness information generated by a plurality of clinical decision support (“CDS”) systems, the method comprising:
receiving input;
identifying, with an electronic processor, a set of CDS systems to access from the plurality of CDS systems;
for each CDS system included in the set of CDS systems,
establishing, with the electronic processor, electronic communication with the CDS system,
submitting, with the electronic processor, a request for appropriateness information to the CDS system based on the input, and
receiving, with the electronic processor, appropriateness information from the CDS system; and
aggregating, with the electronic processor, the appropriateness information received from each CDS system included in the set of CDS systems to generate an aggregated list and displaying the aggregated list on a display device.

3. The method of claim 2, wherein receiving the input includes receiving clinical data for a patient and order data specifying a medical procedure ordered for the patient.

4. The method of claim 3, wherein receiving the appropriateness information from the CDS system includes receiving an indicator of an appropriateness of the medical procedure ordered for the patient based on the clinical data.

5. The method of claim 3, wherein receiving the appropriateness information from the CDS system includes receiving a recommended medical procedure for the patient based on the clinical data.

6. The method of claim 2, wherein receiving the input includes automatically receiving the input from a clinical data collection system.

7. The method of claim 2, wherein receiving the input includes displaying a graphical user interface for receiving the input from a user.

8. The method of claim 2, wherein receiving the input includes receiving at least a portion of the input from a user and wherein identifying the set of CDS systems to access from the plurality of CDS systems includes identifying one or more of the plurality of CDS systems based on an identify of the user or a group the user belongs to.

9. The method of claim 2, wherein identifying the set of CDS systems to access from the plurality of CDS systems includes identifying the set of CDS systems based on configurable parameters.

10. The method of claim 2, wherein identifying the set of CDS systems to access from the plurality of CDS systems includes identifying the set of CDS systems based on manual selections received from a user through a graphical user interface.

11. The method of claim 2, wherein establishing electronic communication with the CDS system includes accessing a list of the plurality of CDS systems stored in memory including data for accessing each of the plurality of CDS systems, identifying each CDS system included in the set of CDS systems within the list, and identifying the data for accessing each of the plurality of CDS systems within the list.

12. The method of claim 2, wherein establishing the electronic communication with the CDS system includes accessing a portal associated with the CDS system over a communication network, the portal including a web page accessible through a browser application.

13. The method of claim 12, wherein accessing the portal associated with the CDS system over the communication network includes automatically entering credentials for accessing the CDS system through the web page.

14. The method of claim 2, wherein aggregating the appropriateness information received from each CDS system included in the set of CDS systems to generate the aggregated list includes including the appropriateness information received from each CDS system included in the set of CDS systems and an identifier of the CDS system providing the appropriateness information in the aggregated list.

15. The method of claim 2, wherein aggregating the appropriateness information received from each CDS system included in the set of CDS systems to generate the aggregated list includes generating a score based on the appropriateness information received from each CDS system included in the set of CDS systems.

16. The method of claim 2, further comprising comparing the aggregated list to a threshold to identify whether to approve a medical procedure ordered for a patient.

17. The method of claim 2, wherein displaying the aggregated list on the display device includes transmitting the aggregated list to an external computing device over a communication network, the external computing device displaying the aggregated list on a display device associated with the external computing device.

18. The method of claim 2, further comprising receiving a selection of one CDS system included in the set of CDS systems and identifying a preferred CDS system of the plurality of CDS systems based on the selection.

19. The method of claim 2, wherein aggregating the appropriateness information received from each CDS system included in the set of CDS systems to generate the aggregated list includes ranking the appropriateness information received from each CDS system of the set of CDS systems.

20. One or more non-transitory machine-readable mediums containing instructions that, when executed by one or more electronic processors, perform a set of functions comprising:
automatically receiving input from a clinical data collection system, the input including clinical data for a patient and order data specifying a medical procedure ordered for the patient;
identifying a configurable set of clinical decision support (“CDS”) systems to access from a plurality of CDS systems;
for each CDS system included in the set of CDS systems,
establishing electronic communication with the CDS system through a portal included in the CDS system over a communication network,
submitting a request for appropriateness information to the CDS system based on the input, and
receiving appropriateness information from the CDS system, the appropriateness information includes at least one selected from the group consisting of an indicator of an appropriateness of the medical procedure ordered for the patient based on the clinical data and a recommended medical procedure for the patient based on the clinical data; and
aggregating the appropriateness information received from each CDS system included in the set of CDS systems to generate an aggregated list and displaying the aggregated list on a display device, the aggregated list including a ranking of the appropriateness received from each CDS system included in the set of CDS systems.
